(19) United States
(12) Patent Application Publication
(10) Pub. No.: US 2008/0069867 A1
(43) Pub. Date: Mar. 20, 2008

(54) Diagnostic and/or Remedy for Ovarian Cancer

(75) Inventors: Kaoru Shimada, Chiba (JP); Yasuhiro Matsumura, Tokyo (JP); Toshiaki Tagawa, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Mitsubishi Pharma Corporation, Osaka-shi (JP)

(21) Appl. No.: 11/575,406

(22) PCT Filed: Aug. 19, 2005
(86) PCT No.: PCT/JP05/15119

§ 371 Date: Oct. 1, 2007

(30) Foreign Application Priority Data
Sep. 16, 2004 (JP) 2004-269757

(51) Int. Cl. A61K 9/127; A61K 39/395; A61P 37/00

(52) U.S. Cl. 424/450; 424/130.1

(57) ABSTRACT

It was found that an antibody containing the amino acid sequences described in the sequence listing as SEQ ID NOS: 1 to 6 was an antibody having reactivity also to ovarian cancer, as well as the cancer species known so far such as gastric cancer, colon cancer and breast cancer, and was useful as an ovarian cancer tissue-specific diagnostic and/or therapeutic agent.

### TECHNICAL FIELD

The present invention relates to a diagnostic and/or therapeutic agent for ovarian cancer.

### BACKGROUND ART

In the fields of diagnosis and therapeutic treatment of cancers, researches are being made on targeting therapy aiming at specific cancer cells for reducing adverse reactions of therapeutic agents, as well as obtaining sufficient curative effect. Specifically, examples include a method of identifying an antigen that is specifically expressed on the surface of cancerous cells, obtaining a monoclonal antibody recognizing the antigen, and using the monoclonal antibody as a therapeutic agent for the cancer (Non-patent document 1 or Non-patent document 2).

Numbers of reports were made on monoclonal antibodies that are usable for the targeting therapy, and an example includes GAHH antibody which is a human monoclonal antibody screened on the basis of reactivity with gastric cancer and colon cancer (Patent document 1).

The GAH antibody is known to exhibit reactivity also to breast cancer in addition to gastric cancer and colon cancer used for the screening, however, it does not exhibit reactivity to lung cancer (Patent document 2). It is thus considered to be difficult, even for those skilled in the art, to predict reactivity of a monoclonal antibody to different type cancers, and this is attributable to a feature of a monoclonal antibody, i.e., extremely high specificity to an antigen

Human non-muscle type myosin heavy chain type A (nmMHCA) has been identified as an antigen of the GAH antibody. However, to the best knowledge of the inventors of the present invention, no report has been made suggesting that the antigen is strongly expressed in ovarian cancer tissues.
Patent document 1: Japanese Patent Unexamined Publication (Kokai) No. 5-304987
Patent document 2- International Patent Publication WO03/009870
Non-patent document 1: Vogel C., Cobleigh M. A Eur. J. Cancer, 2001 January, 37, Suppl. 1:25-29
Non-patent document 2: J. Baselga, Clinical Trials of Herceptin, Eur. J. Cancer, 37, Suppl. 1 (2001) S18-24
Non-patent document 3- Hosokawa S., Hybridoma And Hybridomics, vol. 23, No. 2 (2004) 109-120

### DISCLOSURE OF THE INVENTION

### Object to be Achieved by the Invention

An object of the present invention is to provide a diagnostic and/or therapeutic agent useful for ovarian cancer.

### Means for Achieving the Object

The inventors of the present invention found that an antibody containing the amino acid sequence described in the sequence listing, as any one of SEQ ID NOS- 1 to 6, was an antibody having reactivity to ovarian cancer, additionally to the cancer species known so far such as gastric cancer, colon cancer, and breast cancer, and was useful as an ovarian cancer tissue-specific diagnostic and/or therapeutic agent, and accomplished the present invention.

Gists of the present invention are as follows.
1. A diagnostic and/or therapeutic agent for ovarian cancer, which comprises an antibody containing the amino acid sequence of any one of SEQ ID NOS: 1 to 6 described in the sequence listing.
2. The diagnostic and/or therapeutic agent for ovarian cancer according to 1, wherein the antibody contains the amino acid sequences of SEQ ID NOS: 1, 2 and 3 described in the sequence listing in the hypervariable region of the heavy chain, and the amino acid sequences of SEQ ID NOS: 4, 5 and 6 described in the sequence listing in the hypervariabie region of the light chain.
3. The diagnostic and/or therapeutic agent for ovarian cancer according to 1 or 2, wherein the antibody contains the amino acid sequence of SEQ ID NO: 7 described in the sequence listing in the heavy chain variable region, and the amino acid sequence of SEQ ID NO: 8 described in the sequence listing in the light chain variable region.
4. A diagnostic and/or therapeutic agent for ovarian cancer, which comprises a liposome encapsulating the antibody according to any one of 1 to 3 and a medicament.
5. The diagnostic and/or therapeutic agent for ovarian cancer according to 4, wherein the antibody binds to the liposome via thioether group
6. The diagnostic and/or therapeutic agent for ovarian cancer according to 4 or 5, wherein the antibody binds to the liposome in which a part of lipid ends is maleimidated via a thioether group.
7. The diagnostic and/or therapeutic agent for ovarian cancer according to 6, wherein the antibody binds in an amount of 0.1 to 2 mole % based on the molar amount of the maleimidated lipid.
8. The diagnostic and/or therapeutic agent for ovarian cancer according to any one of 4 to 7, wherein the antibody is an F(ab′)₂ fragment.
9. The diagnostic and/or therapeutic agent for ovarian cancer according to 4, wherein the liposome binds a compound containing a polyalkylene glycol moiety.
10. The diagnostic and/or therapeutic agent for ovarian cancer according to 9, wherein the compound containing a polyalkylene glycol moiety binds in an amount of 15 to 50 mole % based on the molar amount of the maleimidated lipid contained in the liposome.
11. The diagnostic and/or therapeutic agent for ovarian cancer according to 9 or 10, wherein the compound containing a polyalkylene glycol moiety is a compound having two polyalkylene glycol moieties.
12. The diagnostic and/or therapeutic agent for ovarian cancer according to any one of 9 to 11, wherein the polyalkylene glycol moiety is derived from a polyethylene glycol.
13. The diagnostic and/or therapeutic agent for ovarian cancer according to 12, wherein the polyethylene glycol has a molecular weight of 2000 to 7000 daltons.
14. The diagnostic and/or therapeutic agent for ovarian cancer according to 4, wherein the medicament is an antitumor substance.

### Effect of the Invention

According to the present invention, a diagnostic and/or therapeutic agent useful for ovarian cancer can be provided.

### BRIEF DESCRIPTION OF THE DRAWINGS

FIG. 1 shows immnunohistological staining images of tissues of human ovarian cancer patients (A, C) and human non-cancerous ovarian tissues (B, D) obtained by using the GAH antibody.

FIG. 2 shows immunohistological staining images of human ovarian cancer tissues obtained by using the GAH antibody A) or a human IgG antibody (B).

FIG. 3 shows typical staining images of ovarian cancer of serous adenocarcinoma (A), endometrioid carcinoma (B), clear cell cancer (C), and mucinous adenocarcinoma (D) histological types stained with the GAH antibody.

FIG. 4 shows in vitro antitumor effect of GCAH antibody-bound liposomes encapsulating DXR on human ovarian cancer cells. The vertical axis represents ratio (%) of the number of cells for each treatment concentration relative to that of the control (medicament concentration: 0). The horizontal axis represents concentration of the antibody-bound liposomes (in terms of DXR concentration (μ/ml)).

### BEST MODE FOR CARRYING OUT THE INVENTION

In the present invention, the antibody refers to an antibody containing any one of the amino acid sequences of SEQ ID NOS: 1 to 6 described in the sequence listing. Preferred examples thereof include an antibody containing, in the hypervariable region of the heavy chain, the amino acid sequences of SEQ ID NOS: 1, 2 and 3 described in the sequence listing, and containing in the hypervariable region of the light chain, the amino acid sequences of SEQ ID NOS: 4, 5 and 6 described in the sequence listing, and particularly preferred examples include an antibody of which heavy chain variable region and light chain variable region consist of the amino acid sequences of SEQ ID NOS: 7 and 8 described in the sequence listing, respectively.

The hypervariable region mentioned above determines the specificity of immunoglobulin as an antibody and binding affinity between an antigenic determinant and the antibody, and is also called as a complementarity determining region. Therefore, according to the present invention, regions other than the hypervariable region may be derived from other antibodies More specifically, it is understood that any of antibodies containing the hypervariable regions fall within the scope of the antibody of the present invention.

Further, any antibodies modified by substitution, insertion, deletion, addition or the like of a part of amino acid residues not degrading the reactivity to ovarian cancer tissues also fall within the scope of the antibody of the present invention

Preferred examples of the antibody include m onoconal antibodies, and particularly preferred examples include human m onclonal antibodies. When the diagnostic and/or therapeutic agent for ovarian cancer of the present invention is administered to a human, human monoclonal antibodies are advantageous in that they are not proteins derived from heterogenous animals.

The human monoclonal antibodies can be obtained by, for example, producing hybridomas of lymphocytes derived from a cancer patient and mouse myeloma cells, and selecting an antibody having the aforementioned specific amino acid sequences.

Hybridomas can be obtained according to the method of A. Imam et al [Cancer Research, 45, 263 (1985)] by first isolating lymphocytes from a lymph node belonging to cancer extracted from a cancer patient and fusing them with mouse myeloma cells using polyethylene glycol. Cloning is performed by selecting hybridomas that produce antibodies exhibiting positive results for paraformaldehyde-fixed various cancer cell strains in enzyme immunoassays using the supernatants of the resulting hybridomas Then, monoclonal antibodies are purified from the supernatants of hybridomas in a conventional manner [R. C. Duhamel et al, J. Immunol. Methods, 31, 211 (1979)], and labeled with a fluorescent substance, and antibodies exhibiting reactivity to live cancer cell strains, but not exhibiting reactivity to erythrocytes and leucocytes are selected by detecting the reactivities to living cancer cell strains, various kinds of erythrocytes, leucocytes, and the like by means of flow cytometry. Alternatively, by comparing reactivities of antibodies to cancer cells isolated from a cancer tissue extracted from a cancer patient and nor al cells isolated from a nor-cancerous portion of the same patient, antibodies binding to the Cancer cells in a larger amount and exhibiting no reactivity to the normal cells or reactivity in a degree similar to that of antibodies derived from a healthy subject are selected.

Nucleotide sequences of DNAs encoding antibodies produced by hybridomas selected as described above can be obtained by, for example, the following methods. From antibody-producing hybridomas, mRNAs are prepared by the guanidine thiocyanate lithium chloride method [Casara et al., DNA, 2, 329 (1983)], and a CDNA library thereof is prepared by using oligo(dT) primers. Then, (dG)-tailing is performed for the cDNAs, and PCR is performed by using a poly C that hybridizes with that dg-tail and a portion of sequence shared by the already obtained human antibody heavy chain gene and light chain gene as probes to amplity cDNAs encoding antibodies. Then, DNAs are blunt-ended, and subjected to electrophoresis, DNAs contained in bands excised from gel are inserted into a cloning vector such as pULC119, and the nucleotide sequences thereof are determined by the dideoxy method of Sanger et al. [Proc Natl. Acad. Sci U.S.A., 74, 5463 (1977)]. On the basis of these nucleotide sequences, antibodies having the aforementioned specific amino acid sequences can be selected.

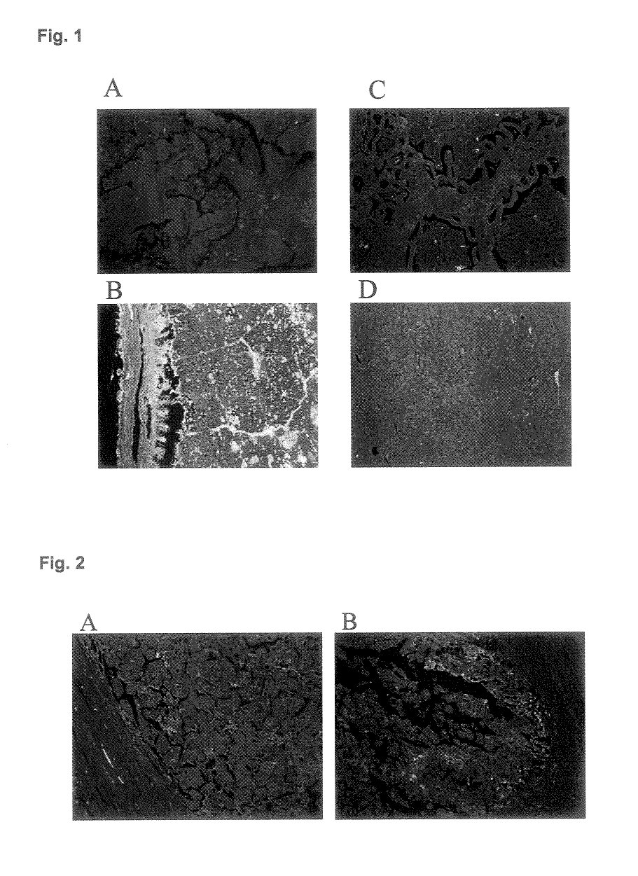

The antibody can also be prepared by a genetic engineering technique. Although methods used in the preparation are not particularly limited, examples include the following methods. The antibody can be obtained by culturing a hybridoma producing the antibody in a medium such as eRDF containing fetal bovine serum and RPMI 1640 medium, or chemically synthesizing genes comprising DNA encoding a variable region including the aforementioned specific hypervariable region further ligated with each of the constant regions of the heavy chain and light chain, inserting the genes into any of known various expression vectors that can express the genes, for example, as expression vectors in animal cells, pKCR(Δ E)/H, which can be constructed from pKCRH2 [Mishina et al., Nature, 307, 605 (1984)] according to the procedure shown in Japanese Patent Unexamined Publication No. 5-304987, FIGS. 1 or 2, and pKCRD, and expressing the genes in a host such as the CHO cells (Chinese hamster ovarian cells). For example, the heavy chain gene added with the HindIII sites at both ends is inserted into the HindIII site of pKCR(Δ E)/H, and a selection marker gene such as the DHFF gene is also inserted into the SalI site of that plasmid Separately, the light chain gene added with the EcoRI sites at both ends is inserted into the EcoRI site of pKCRD, and the DHFR gene is further inserted also into the SalI site of this plasmid. Both of the plasmids are introduced into cells, such as CHOdhfr- [Urlaub G. & Chasin L. A., Proc. Natl. Acad. Sci. U.S.A, 77, 4216 (1980)], by the calcium phosphate method, and cells producing the antibody can be obtained by selecting the cells from cells proliferating in α MEM medium not containing nucleotides, or the like. The antibody is purified from a medium in which these cells were cultured, for example, by adsorbing the antibody on a column containing a support such as Cellulofine and agarose bound with protein A and then eluting the antibody.

The nucleotide sequences of the constant regions of the heavy chain and light chain of the antibody may be those having the same sequences as those described in, for example, Nucleic Acids Research, 14, 1779 (1986); The Journal of Biological Chemistry, 257, 1516 (1982); and Cell 22, 197 (1980).

The antibody includes a full length antibody (whole antibody), an antibody fragment (antibody fragment such as Fab′, F(ab′)₂ and scfv (single chain antibody)), a derivatized antibody, a modified antibody, and the like, and the term should be construed in the broadest sense. Most preferably, an example includes an F(ab′)₂ fragment.

The antibody may be independently used, or may be used as a conjugate with a medicament, or the like. Examples of the method for conjugating a medicament and the antibody include a method of directly binding a medicament to the antibody, a method of binding a medicament by means of a spacer, a method of binding a liposome encapsulating a medicament to the antibody, and the like The method of binding a liposome encapsulating a medicament to the antibody is preferred.

According to the present invention, the liposome is constituted by lipids, for example, but not limited to, natural lecithins (for example, egg yolk lecithin, soybean lecithin), phospholipids such as dipalmitoylphosphatidylcholine (DPPC), dimyristoylphosphatidylcholine (DMPC), distearoylphosphatidylcholine (DSPC), dioleoylphosphatidylcholine (DOPC), dimyristoylphosphatidylethanolamine (DMPE), dip almitoylphosphatidylethanolamine (DPPE), dioleoylphosphatidylethanolamine (DOPE), dipalmitoylphosphatidic acid (DPPA), dipalmitoylphosphatidylglycerol (DPPG) and dimyristoylphosphatidic acid (DMPA), glycolipids such as sphingoglycolipids and glycoglycerolipids, aliphatic acids, dialkyl dimethylammuonium amphiphiles, polyglycerol alkyl ethers, polyoxyethylene alkyl ethers, and the like (Liposome Technology, 2nd edition, vol 1, 141, 1993), alkyl glycosides, alkylmethyl glucamides, alkyl sucrose esters, dialkyl polyoxyethylene ethers, dialkyl polyglycerol ethers (Liposome Technology, 2nd edition, vol. 1, 141, 1993), and the like, amphiphilic block copolymers such as polyoxyethylene/polylactic acid (Japanese Patent Unexamined Publication based on International Patent Application (Kohyo) No. 6-508831), and the like. These lipids may be used independently or two or more kinds of lipids may be used in combination. Further, they may be used in combination with nonpolar substances such as cholesterol and cholesterol derivatives such as DC-chol (3β-[N-(N′,N′-dimethylaminoethyl)carbamoyl]cholesterol).

The method for preparing the liposome is not particularly limited, and any methods available for those skilled in the art may be used. A form of the liposome is not particularly limited, and any kind of form may be used. The liposome may be in the form of, for example, any of multilamellar vesicle (MLV), which is formed by adding an aqueous solution to a lipid thin membrane adhered to a glass wall and adding mechanical vibration; small unilamellar vesicle (SUV), which is obtained by the ultrasonication method, ethanol injection method or French press method; and large unilamellar vesicle (LUV), which is obtained by the surfactant removing method, reverse phase evaporation method (Liposome, Junzo Sunamoto et al., Nankodo, 1998), or extrusion method in which MLVs are extruded through a membrane having pores of uniform diameters by pressurization (Liposome Technology, 2nd edition, vol. 1, 141, 1993) The liposome has a diameter of, for example, about 300 nm or smaller, preferably about 30 to 200 nm.

According to the present invention, the method for introducing a medicament into the liposome is not particularly limited, and any of the methods available for those skilled in the art may be used. For example, a medicament may be encapsulated in the liposome by adding the medicament as an aqueous solution at the time of liposome formation. Further, also employable methods include a method of, after formation of liposomes, forming a concentration gradient such as a pH gradient for the outside and inside of the vesicles and incorporating an ionizable antitumor agent using the gradient as a driving force (Cancer Res., 49, 5922, 1989; BBA, 455, 269, 1976), and the like.

According to the present invention, although the method of binding the antibody to the liposome is not particularly limited, the antibody is preferably bound to the surface of the liposome. Examples of the method include a method of binding a hydrophobic substance to a purified antibody and inserting the antibody into a liposome, a method of crosslinking phosphatidylethanolamine and the antibody with glutal, and the like, and preferred examples include a method of modifying a liposome with the antibody by adding thiol group to the antibody and then reacting maleimide group of the liposome and the thiolated antibody. The addition of thiol group to the antibody can be attained by a method of reacting a compound commonly used for thiolation of a protein such as N-succinimidyl-3-(2-pyridylthio)propionate (SPDP, Carlsson, J., et al., Biochem. J., 173, 723, 1978), iminothiolane, and mercaptoalkylimidate (Traut, R. R., et al, Biochemistry, 12, 3266, 1973) with amino group of the antibody.

Further, a sulfur-containing group derived from the antibody, i.e., an inherent diol group of the antibody, may also be reacted, and a method of using inherent dithiol group is preferred from a viewpoint of maintenance of activity of the antibody. The inherent dithiol group of the antibody can be reduced into thiol groups, and then reacted with maleimide group. For example, when IgG is used, the antibody may be made into F(ab′)₂ with an enzyme such as pepsin and further made into Fab′ by reduction of F(ab′)₂ with dithiothreitol or the like, and thiol group generated in Fab′ can be used for binding to a liposome (Martin, F. J., et al, Biochemistry, 20, 4229, 1981). As for IgM, thiol group of the Fc moiety of IgMs obtained by reducing the J-chain under a mild condition according to the method of Miller et al (J. Biol. Chem., 257, 286, 1965) may be used for the binding to a liposome. When the GAH antibody described in Japanese Patent Unexamined Publication No. 5-304987 is used, it is preferable to use F(ab′)₂. The binding of a protein such as antibody added with thiol group and a liposome containing maleimide group is usually attained by reacting them in a neutral buffer (pH 6.5 to 7.5) for 2 to 16 hours.

According to the present invention, the amount of the antibody binding to the liposome is, for example, about 0.1 to 2 mole %, preferably 0.1 to 1.6 mole %, more preferably 0.4 to 0.7 mole %, based on the molar amount of maleimidated lipid.

According to the present invention, the liposome is not particularly limited. The liposome preferably has a form in which the aforementioned compound containing a polyalkylene glycol moiety binds to a maleimidated lipid on the surface of the liposome via thioether bond. Examples of the compound containing a polyalkylene glycol moiety include a compound having a polyethylene glycol group and an end that can be thiolated, and a compound having a polyethylene glycol group and mercapto group at the end. Specific examples include, for example, a compound comprising a polyalkylene glycol group bound to triazine, and a compound comprising a polyalkylene glycol group bound to triazine substituted with an amino acid or the like. These compounds may be compounds having two polyalkylene glycol groups (double chain)

According to the present invention, the binding amount of the compound having a polyalkylene glycol moiety to the liposome is not particularly limited, and may be reacted in an excessive amount with respect to residual maleimidated lipid. However, the binding amount of the polyalkylene glycol is preferably about 0.28 to 0.90 mole %, more preferably about 0.28 to 0.56 mole %, with respect to the total lipid, preferably about 15 to 50 mole %, more preferably about 15 to 30 mole %, with respect to maleimidated lipid, or preferably about 0.44 to 1.45 mole %, more preferably about 0.44 to 0.89 mole %, with respect to DPPC.

Examples of the polyalkylene glycol referred to in the present invention include, for example, polyethylene glycol (PEG), polypropylene glycol, and the like, and preferred examples include polyethylene glycol. When a polyethylene glycol is used, those having a molecular weight of about 2,000 to 7,000 daltons are preferred, and those having a molecular weight of about 5,000 daltons are most preferred.

When the aforementioned compound having a polyalkylene glycol moiety has a form of binding to a maleimidated lipid on the surface of the liposome by means of thioether bond, a liposome bound with a polyalkylene glycol can usually be prepared by introducing thiol group into the compound having a polyalkylene glycol moiety, and then reacting the resulting compound with the maleimide group of the liposome.

For preparing the compound having a polyalkylene glycol moiety introduced with thiol group by using a polyethylene glycol as the polyalkylene glycol, usable methods include, for example, a method of performing dehydration condensation of monomethoxypolyoxyethyleneamine and any of various thiolcarboxylic acid; a method of introducing pyridyldithiopropionyl group into monomethoxypolyoxyethyleneamine using SPDP and further reducing the resultant; a method of introducing thiol group into monomethoxypolyoxyethyleneamine by using iminothiolane; a method of binding any of various thiolamines to an active ester of monomethoxypolyoxyethylenecarboxylic acid; a method of condensing a polyethylene glycol triazine derivative with thiolamine, and the like. Still more specifically, 2,4-bis-(polyethylene glycol)-6-chloro-s-triazine (activated PEG2, Seikagaku Corporation) can be reacted with cystine, and the resultant can be reduced to obtain cysteine-bound activated PEG2.

In a preferred embodiment of the present invention, a liposome bound with the antibody and the compound containing a polyalkylene glycol moiety is used, and in order to prepare this kind of liposome, a liposome having maleimide group is first reacted with a thiolated antibody in a neutral buffer. For example, the reaction may be performed so that 0.5 to 5.3 mg, preferably 0.5 to 4.5 mg, more preferably 1.2 to 2 mg, of the antibody binds per 100 mg of the total lipid, i.e., the thiolated antibody may be reacted in an amount of about 0.1 to 2 mole %, preferably 0.1 to 1.6 mole %, more preferably 0.4 to 0.1 mole %, based on the molar amount of maleimide group (maleimidated lipid). Then, the compound having a thiolated polyalkylene glycol moiety can be reacted with the residual maleimide group to prepare a liposome bound with the antibody and the compound containing a polyalkylene glycol moiety. Specifically, by adding 15 to 50 mole %, preferably 15 to 30 mole %, based on the molar amount of maleimidated lipid groups (0.28 to 0.90 mole %, preferably 0.28 to 0.56 mole %, with respect to the total lipids, or when DPPC is used, 0.44 to 1.45 mole %, preferably 0.44 to 0.89 mole%, with respect to DPPC) of the compound having a thiolated polyalkylene glycol moiety, a liposome bound with the antibody and the compound containing a polyalkylene glycol moiety can be prepared.

According to the present invention, examples of the medicament include diagnostic agents and antitumor substances.

According to the present invention, examples of the diagnostic agents include radioactive elements such as indium and technetium, contrast media such as gadolinium and iodine, and the like.

Although type of the antitumor substances is not particularly limited in the present invention for example, antitumor agents (anticancer agents) such as doxorubicin (adriamycin), daunomycin, vinblastine, cisplatin, nedaplatin, and 5-fluorouracil (5-FU); radioactive substances such as iodine-131; toxins such as ricin A and diphtheria toxin; antisense RNAs; pharmacologically acceptable salts and derivatives thereof, and the like can be used. These substances can be obtained by purchasing commercial products or each substance can be suitably prepared according to a known method.

As the aforementioned pharmaceutically acceptable salts, salts with a pharmaceutically acceptable multivalent anionic substance such as citrates, tartrates. glutamates and salts with derivatives thereof are preferred.

The medicament-containing liposome bound with the antibody can be made into a pharmaceutical preparation by a known method, for example, the dehydration method (WO88/06441), the method of adding a stabilizer for use as a solution (Japanese Patent Unexamined Publication No. 64-9931), the lyophilization method (Japanese Patent Unexamined Publication No. 64-9931), and the like, and can be administered to a patient by intravascular administration, topical administration, or the like for diagnosis and/or therapeutic treatment of ovarian cancer. Although the dose can be suitably chosen according to type of an antitumor substance as an active ingredient, when a liposome encapsulating doxorubicin is administered for therapeutic treatment of ovarian cancer, for example, the liposome can be administered in an amount of 50 mg/Kg or less, preferably 10 mg/kg or less, more preferably 5 mg/kg or less, in terms of the amount of the active ingredient.

### EXAMPLES

The present invention will be specifically explained with reference to examples. However, the present invention is not limited to the following examples, unless the examples beyond the gist of the present invention.

### Example 1

An antibody containing the amino acid sequences described in the sequence listing as SEQ ID NOS: 1 to 6 described in Japanese Patent Unexamined Publication No. 5-304987, Examples 1, 2 and 3 (henceforth also abbreviated as GAH antibody) was labeled by biotinylation using a biotinylation reagent Amersham). Paraffin sections of human ovarian cancer tissues (obtained from National Cancer Center Hospital, Pathological Section) and tissues of non-cancerous portions around the cancer tissues (obtained from National Cancer Center Hospital, Pathological Section) were deparaffinized, subjected to a blocking treatment by immersing the samples in a 5% BSAIPBS solution at room temperature for 1 hour, and then reacted with a 100 μg/ml biotinylated GAH antibody solution at 37° C. for 2 hours. The sections were washed with PBS, and reacted with a 4 μg/ml PerCP (peridinine chlorophyll protein)-labeled streptavidin solution (Becton Dickinson) for 30 minutes under light shielding and ice cooling. The reactivity of the GAH antibody to the ovarian cancer tissue sections was detected as red fluorescence of PerCP at 680 nm obtained with an excitation wavelength of 490 nm by using a fluorescence microscope.

The results are shown in FIG. 1. The ovarian cancer tissue sections exhibited definite red stained images representing GAH-positive results as shown in A and C, whereas the reaction of the GAH antibodies was not observed, and thus red stained images were not observed for the non-cancerous tissues of B and D.

### Example 2

As control antibodies, biotinylated antibodies were prepared in the same manner as that used in Example 1 by using human immunoglobulin (IgG), and the human ovarian cancer tissue sections were stained by using the CAAH antibodies and the control antibodies (FIG. 2)

As a result, an intense positive reaction as shown in red stained images obtained with the GAH antibodies (red staining) was not observed with the control antibodies.

### Example 3

The human ovarian cancer tissue sections were reacted with the GAH antibodies in the same manner as that used in Example 1, and the results were determined to be positive or negative on the basis of intensity and distribution of red fluorescence.

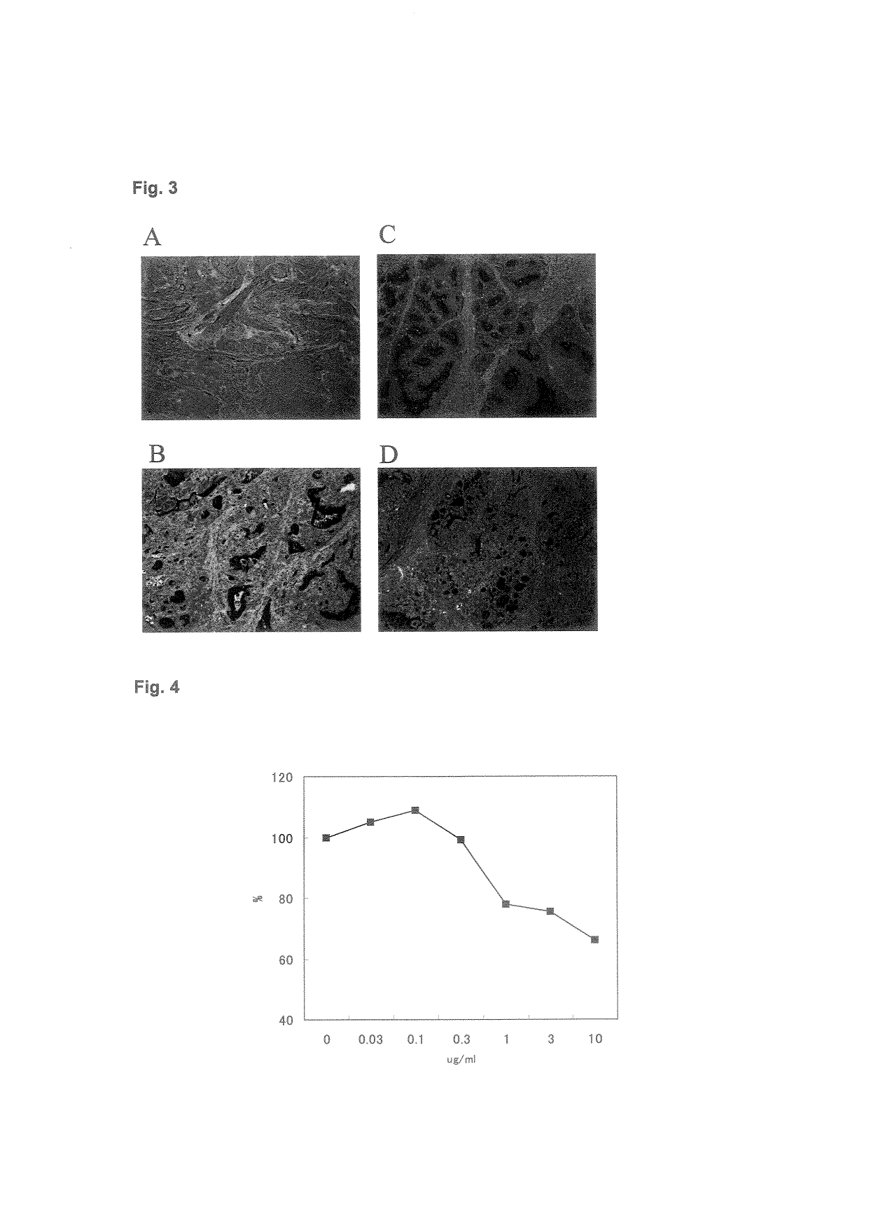

As a result, the GAH antibodies stained 57 sections out of 62 human ovarian cancer tissue sections. Typical examples are shown in FIG. 3 (A: serous adenocarcinoma, B: endometrioid carcinoma, C: clear cell cancer, D: mucinous adenocarcinoma).

From the results mentioned above, it was revealed that the GAH antibodies specifically reacted with human ovarian cancer tissues.

### Example 4

Liposomes encapsulating doxorubicin (DXR, KYOWA HAKKO KOGYO) were prepared according to the method described in WO00/64413 (Example 1). The resulting liposomes encapsulating DXR were bound with thiolated GAH antibodies, and further bound with thiolated polyethylene glycol (PEG) to prepare GAH antibody-bound liposomes

In vitro antitumor effect of the aforementioned antibody-bound immunoliposomes was examined by using human ovarian cancer cells ES-2 (ATCC NO. CRL-1978). To the cells of the strain inoculated on a cell culture plate, the antibody-bound liposomes were added at the concentrations shown in FIG. 4 (indicated in terms of the DXR concentration (μg/ml)). After culture for 48 hours, the medicament was removed, and cell number was counted by the MTT method (Green, L. M., et al., J. Immunol. Methods, 70:257-268, 1984).

As a result, the number of cells decreased as the concentration of the added antibody-bound liposomes increased.

From the results mentioned above, it was revealed that the GAH antibody-bound liposomes had a suppressing effect against ovarian cancer proliferation

### INDUSTRIAL APPLICABILITY

According to the present invention, a prophylactic and/or therapeutic agent also effective for ovarian cancer, as well as gastric cancer, colon cancer and breast cancer, on the basis of a specific reactivity of the antibody can be provided.

This application was filed with claiming the conventional priority based on Japanese Patent Application No. 2004-269757.

1-14. (canceled)

15: A diagnostic and/or therapeutic agent for ovarian cancer, which com rises an antibody containing the amino acid sequence of any one of SEQ ID NOS: 1 to 6 described in the sequence listing.

16: The diagnostic and/or therapeutic agent for ovarian cancer according to claim 15, wherein the ant-body contains the amino acid sequences of SEQ ID NOS: 1, 2 and 3 described in the sequence listing in a hypervariable region of a heavy chain, and the amino acid sequences of SEQ ID NOS: 4, 5 and 6 described in the sequence listing in a hypervariable region of a light chain.

17: The diagnostic and/or therapeutic agent for ovarian cancer according to claim 15, wherein he antibody contains the amino acid sequence of SEQ ID NO: 7 described in the sequence listing in a heavy chain variable region, a amino acid sequence of SEQ ID NO: 8 described in the sequence listing in a light chain variable region.

18: The diagnostic and/or therapeutic agent for ovarian cancer according to claim 16, wherein the antibody contains the amino acid sequence of SEQ ID NO: 7 described in the sequence listing in a heavy chain variable region, and the amino acid sequence of SEQ ID NO: 8 described in the sequence listing in a light chair variable region.

19: A diagnostic and/or therapeutic agent for ovarian cancer, which comprises the antibody according to claim 15 and a liposome encapsulating a medicament.

20: The diagnostic and/or therapeutic agent for ovarian cancer according to claim 19, wherein the antibody binds to the liposome by means of a thioether group.

21: The diagnostic and/or therapeutic agent for ovarian cancer according to claim 19, wherein the antibody binds to the liposome in which a part of lipid ends is maleimidated by means of a thioether group.

22: The diagnostic and/or therapeutic agent for ovarian cancer according to claim 20, wherein the antibody binds to the liposome in which a part of lipid ends is maleimidated by means of a thioether group.

23: The diagnostic and/or therapeutic agent for ovarian cancer according to claim 21, wherein the antibody binds in an amount of 0.1 to 2 mole % based on a molar amount of the maleimidated lipid.

24: The diagnostic and/or therapeutic agent for ovarian cancer according to claim 22, wherein the antibody binds in an amount of 0.1 to 2 mole % based on a molar amount of the maleimodated lipid.

25: The diagnostic and/or therapeutic agent for ovarian cancer according to claim 19, wherein the antibody is an F(ab′)₂ fragment.

26: The diagnostic and/or therapeutic agent for ovarian cancer according to claim 19, wherein the liposome binds a compound containing a polyalkylene glycol moiety.

27: The diagnostic and/or therapeutic agent for ovarian cancer according to claim 26, wherein the compound containing a polyalkylene glycol moiety binds in an amount of 15 to 50 mole % based on a molar amount of the maleimidated lipid contained in the liposome.

28: The diagnostic and/or therapeutic agent for ovarian cancer according to claim 26, wherein the compound containing a polyalkylene glycol moiety is a compound having two polyalkylene glycol moieties.

29: The diagnostic and/or therapeutic agent for ovarian cancer according to claim 27, wherein the compound containing a polyalkylene glycol moiety is a compound having two polyalkylene glycol moieties.

30: The diagnostic and/or therapeutic agent for ovarian cancer according to claim 26, wherein the polyalkylene glycol moiety is derived from a polyethylene glycol.

31: The diagnostic and/or therapeutic agent for ovarian cancer according to claim 30, wherein the polyethylene has a molecular weight of 2000 to 7000 daltons.

32: The diagnostic and/or therapeutic agent for ovarian cancer according to claim 19, wherein the medicament is an antitumor substance.


SEQUENCE LISTING (8 sequences)

SEQ ID NO: 1 - protein, 9 aa, Homo sapiens
  Inventor Shimada Kaoru, Matsumura Yasutaka, Tagawa Toshiaki
ISSCGFYWN

SEQ ID NO: 2 - protein, 12 aa, Homo sapiens
IGYIYYSGSTYY

SEQ ID NO: 3 - protein, 9 aa, Homo sapiens
STRLRGADY

SEQ ID NO: 4 - protein, 17 aa, Homo sapiens
KSSQSVLYNSNNKKYLA

SEQ ID NO: 5 - protein, 7 aa, Homo sapiens
WASTRES

SEQ ID NO: 6 - protein, 9 aa, Homo sapiens
QQYYSTPWT

SEQ ID NO: 7 - protein, 119 aa, Homo sapiens
QVQLQESGPGLVKPSQTLSLTCTVSGGSISSCGFYWNWIRQHPGKGLEWIGYIYYSGSTYYNPSLKSRVTISLDTSKSQFSLKLSSLTAADTAVYYCARSTRLRGADYWGQGTMVTVSS

SEQ ID NO: 8 - protein, 114 aa, Homo sapiens
DIVMTQSPDSLAVSLGERATINCKSSQSVLYNSNNKKYLAWYQQKPGQPPKLLIYWASTRESGVPDRFSGSGSGTDFTLTISSLQAEDVAVYYCQQYYSTPWTFGQGTKVEIKR